(19) United States
(12) Patent Application Publication
(10) Pub. No.: US 2025/0334562 A1
(43) Pub. Date: Oct. 30, 2025

(54) CALCULATION OF EXTRACTION EFFICIENCY COEFFICIENTS FOR MUD-GAS ANALYSIS

(71) Applicant: Equinor Energy AS, Stavanger (NO)

(72) Inventors: Tao YANG, Stavanger (NO); Alexandra CELY, Sandnes (NO); Knut ULEBERG, Sandnes (NO); Gulnar YERKINKYZY, Sandvika (NO); Sandrine DONNADIEU, STRAUMSGREND (NO)

(73) Assignee: Equinor Energy AS, Stavanger (NO)

(21) Appl. No.: 18/870,306

(22) PCT Filed: May 17, 2023
(86) PCT No.: PCT/NO2023/050114

§ 371 Date: Nov. 27, 2024

(30) Foreign Application Priority Data
May 30, 2022 (GB) 2207990.9

(51) Int. Cl. G01N 33/28; E21B 49/08; G01V 20/00

(52) U.S. Cl. CPC G01N 33/2823; E21B 49/086; G01V 20/00

(57) ABSTRACT

A method of calculating extraction efficiency coefficients for mud-gas analysis includes: generating a simulated output drilling fluid based on a mixture of about 1 wt. % of a reference reservoir fluid and a balance of an input drilling fluid; simulating release of gas from the simulated output drilling fluid under predetermined conditions using an equations-of-state model; and determining an extraction efficiency coefficient for each gas component based on a ratio between the composition of the reference reservoir fluid and the composition of the simulated released gas.

The present invention relates to mud-gas analysis, and particularly to a method of calculating at least one extraction efficiency coefficient for mud-gas analysis.

Fluid typing or identification during drilling is important for many real-time well decisions, like well integrity in overburden, optimal well placement in a reservoir zone, completion strategy, and determining potential sidetrack locations. In addition, such data can be used to improve reservoir management and provide better future drilling targets. It is desirable to identify continuous reservoir fluid typing (i.e. whether the reservoir contains reservoir oil or reservoir gas) without deploying expensive logging tools.

Mud gas logging has been extensively used in the industry to achieve this for many decades. The accuracy of the mud gas data composition improved considerably after the advanced mud gas technology was invented in the 1990s. More recently, a machine learning approach has been developed for prediction of gas-oil ratio, and other reservoir fluid properties, from advanced mud gas data, which has generated good results.

Although reservoir fluid identification from advanced mud-gas data has been a breakthrough in the industry, only a small number of wells today have advanced mud gas data. On the contrary, standard mud-gas data is available for all the wells, and there is no additional cost for standard mud-gas data acquisition. Due to low cost and wide availability for all wells, the business impact is significant to develop an accurate fluid typing method using standard mud-gas data.

A need therefore exists for techniques that can achieve reservoir fluid typing using standard mud-gas data.

Viewed from a first aspect, the present invention provides a method of calculating an extraction efficiency coefficient for mud-gas analysis, the method comprising: providing a composition of an input drilling fluid; providing a composition of a reservoir fluid; generating a composition of a simulated output drilling fluid based on the compositions of the input drilling fluid and the reservoir fluid; simulating release of a selected gas component from the simulated output drilling fluid under predetermined conditions; and determining the extraction efficiency coefficient for the selected gas component based on a ratio between a concentration of the selected gas component within the composition of the reservoir fluid and a simulated concentration of the selected gas component released from the simulated output drilling fluid.

By generating an extraction efficiency coefficient by the method above, it is possible to use only reservoir fluid composition data and drilling fluid composition data, which can both be measured accurately. This avoids problems associated with noise, which is common in mud-gas data, and particularly prevalent in standard mud-gas data.

Furthermore, new extraction efficiency coefficients are required for new mud-gas extraction operational conditions, these can be derived analytically by simply changing the predetermined conditions of the simulated release of gas.

Simulating release of the selected gas component from the simulated output drilling fluid under predetermined conditions may be performed using an equations-of-state model corresponding to the composition of the simulated output drilling fluid.

An equations-of-state model is a fluid model that takes a molar composition of a fluid and predicts the phase split and volumetric behaviour of the fluid (e.g., vapour and liquid phase compositions, densities, viscosities and formation volume factors) over a range of pressures and temperature.

The method may be performed for a plurality of selected gas components. That is to say, the simulating step may comprise simulating release of a plurality of selected gas component from the simulated output drilling fluid under predetermined conditions, and the determining step may comprise determining the extraction efficiency coefficient for each selected gas component. The selected gas components may comprise each of C₁, C₂ and C₃. Optionally, the plurality of selected gas components may additionally comprise each of C₄ and C₅.

The extraction efficiency coefficient may correspond to a specific hydrocarbon field, and the composition of the reservoir fluid may be a composition of a reservoir fluid sample from a hydrocarbon well in the specific hydrocarbon field.

Alternatively, the extraction efficiency coefficient may correspond to a specific hydrocarbon well, and the composition of the reservoir fluid may be a composition of a reservoir fluid sample from the specific hydrocarbon well.

The composition of the reservoir fluid sample may be a measured composition, which may have been collected from a downhole fluid analysis or downhole fluid sampling. The composition of the reservoir fluid sample may be retrieved from a database of reservoir fluid data.

The predetermined conditions may correspond to operational conditions of a mud-gas analysis unit.

The predetermined conditions may comprise at least a predetermined pressure and a predetermined temperature.

The predetermined pressure may be approximately atmospheric pressure, for example between 0.5 bar and 2.0 bar, or between 0.8 bar and 1.5 bar or between 0.9 bar and between 1.1 bar.

The predetermined temperature may be between 0° C. and 100° C., or between 10° C. and 50° C., or between 70° C. and 100° C., or between 80° C. and 90° C.

The simulated output drilling fluid may comprise between 0.01 wt. % and 5 wt. % of the reservoir fluid, or between 0.2 wt. % and 2 wt. % of the reservoir fluid.

The simulated output drilling fluid may comprise at least 50 wt. % of the input drilling fluid, or at least 80 wt. % of the input drilling fluid, or at least 90 wt. % of the input drilling fluid, or at least 95 wt. % of the input drilling fluid. The simulated output drilling fluid may comprise a balance of the input drilling fluid.

The simulated output drilling fluid may comprise up to 5 wt. % of one or more simulated impurities.

Viewed from a second aspect, the present invention provides a method comprising: receiving mud-gas data; and performing an extraction efficiency correction on the mud-gas data to produce corrected mud-gas data, wherein the extraction efficiency correction comprises applying a plurality of extraction efficiency coefficients to the mud-gas data, each extraction efficiency coefficient having been determined by a method as set out above.

The mud-gas data may comprise standard mud-gas data. For example, the mud-gas data may not have had an extraction efficiency correction applied and/or may not have had a recycling correction applied. The mud-gas data may have been collected at a temperature below 50° C.

The method may comprise: identifying one or more geochemical parameter based on the corrected mud-gas data; and identifying a fluid type of a target reservoir fluid based on a threshold associated with the or each geochemical parameter.

The geochemical parameter may be derivable from C₁ to C₃ fluid composition data. Geochemical parameters derived from C₁ to C₃ fluid composition can be determined with reasonable confidence based on standard mud-gas data. Thus, fluid can be accurately typed using standard mud-gas data.

The geochemical parameter may comprise one of: a C₁/C₂ ratio; a C₁/C₃ ratio; and a Bernard parameter, C₁/(C₂+C₃).

In other embodiments, the geochemical parameter may be derivable from C₁ to C₅ fluid composition data. For example, the geochemical parameter may comprise one of: a balance ratio, (C₁+C₂)/(C₃+C₄+C₅); a wetness ratio, (C₂+C₃+C₄+C₅)/(C₁+C₂+C₃+C₄+C₅); a dryness ratio, C₁/(C₁+C₂+C₃+C₄+C₅); and a hydrocarbon character, (C₄+C₅)/(C₃).

The threshold may be a region-specific threshold.

The threshold may be for distinguishing between a first fluid type and a second fluid type within the region of interest. The first fluid type may be reservoir oil and the second fluid type may be reservoir gas.

The method may comprise obtaining reservoir fluid properties data corresponding to a plurality of fluid samples; identifying a fluid type and at least one geochemical parameter for each of the fluid samples that are within the region of interest, and determining the region-specific threshold for the or each geochemical parameter based on the fluid type of the plurality of fluid samples within the region of interest.

The method may comprise determining a threshold confidence for the region-specific threshold associated with the or each geochemical parameter. The threshold confidence may be determined based on the fluid samples that are within the region of interest, and particularly based upon the fluid type and the respective geochemical parameter of each of the fluid samples that are within the region of interest. The threshold confidence may be determined using any suitable statistical method.

The threshold confidence may be indicative of a confidence associated with the region-specific threshold for distinguishing between the first fluid type and the second fluid type within the region of interest. That is to say, a confidence that a corresponding geochemical parameter value for a fluid sample from the region of interest that is below the region-specific threshold will correspond to one of the fluid types, and one that is above the region-specific threshold will correspond to the other of the fluid types.

The threshold confidences may be useful to informing an operator regarding the accuracy of a particular fluid type determination. Furthermore, it may indicate which of the geochemical parameters should be used for a particular region of interest, as not all parameter may provide sufficient accuracy when determining the fluid type.

Whilst the method may be employed using a single geochemical parameter, preferably the one or more geochemical parameter comprises a plurality of geochemical parameters.

The method may further comprise identifying at least one distinguishing geochemical parameter from amongst the one or more geochemical parameter based on the threshold confidences. The at least one distinguishing geochemical parameter may be region-specific, i.e. for distinguishing fluid types within the region of interest. Identifying the fluid type of the target reservoir fluid may be based on the region-specific threshold associated with the or each of the at least one distinguishing geochemical parameter for the target reservoir fluid.

The at least one distinguishing geochemical parameter is preferably a subset of the at least one geochemical parameter. The method may examine multiple geochemical parameters, and select a subset (optionally including all of them if appropriate) based on the threshold confidences. That this to say, the original geochemical parameters may be test geochemical parameters, which may be evaluated to determine the distinguishing geochemical parameters having sufficient confidence for the region of interest. Preferably, those test geochemical parameters having the highest confidences are selected, for example having a threshold confidence above a predetermined threshold.

Optionally, identifying the fluid type of the target reservoir fluid may be further based on a weighting based on the threshold confidences associated with the at least one geochemical parameter. For example, a fluid type indication based on a geochemical parameter having a relatively high confidence may be given greater weight than a fluid type indication based on a geochemical parameter having a relatively low confidence.

Additionally, the threshold confidences may be used to guide an operator regarding what data should be collected.

The method may comprise determining that a threshold confidence associated with a geochemical parameter derivable from C₁ to C₃ fluid composition data is above a predetermined level. Consequently, obtaining the mud-gas data may comprise obtaining standard mud gas data in response to the determination.

This may be advantageous as standard mud-gas data is cheaper to collect that advanced mud-gas data.

The method may comprises determining that a threshold confidence associated with a geochemical parameter derivable from C₁ to C₃ fluid composition data is below a predetermined level. Consequently, obtaining the mud-gas data comprises obtaining advanced mud gas data and/or obtaining standard mud gas using heating in response to the determination. The heating may comprise heating to a temperature of at least 40° C., at least 50° C., at least 70° C., at least 80° C., or at least 90° C.

Using advanced mud gas data and/or heated standard mud gas improves the accuracy of C₄ and C₅ fluid composition data, which allows for a larger number of geochemical parameters to be used when those that can be calculated based on C₁ to C₃ fluid composition data are insufficient.

The mud-gas data may comprise historic mud-gas data, for example where the mud-gas data was collected more than 1 day ago, or more than 1 month ago, or more than 6 months ago. The method is particularly advantageous as it can be applied retrospectively to historic data.

Alternatively, the method may comprise: drilling a well bore using drilling fluid, the well bore passing through a reservoir containing the reservoir fluid; collecting output mud gas from the drilling fluid after passing through the well bore; and measuring a composition of the output mud gas as the mud-gas data.

In a preferred embodiment the method is a computer-implemented method, e.g. the steps of the method are performed by processing circuitry.

The method may be implemented at least partially using software, e.g. computer programs. It will thus be seen that when viewed from further aspects the present invention provides computer software specifically adapted to carry out the methods described herein when installed on a data processor, a computer program element comprising computer software code portions for performing the methods described herein when the program element is run on a data processor, and a computer program comprising code adapted to perform all the steps of a method or of the methods described herein when the program is run on a data processing system.

The present invention also extends to a computer software carrier comprising such software arranged to carry out the steps of the methods of the present invention. Such a computer software carrier could be a physical storage medium such as a ROM chip, CD ROM, DVD, RAM, flash memory or disk, or could be a signal such as an electronic signal over wires, an optical signal or a radio signal such as to a satellite or the like.

The present invention may accordingly suitably be embodied as a computer program product for use with a computer system. Such an implementation may comprise a series of computer readable instructions, which may be fixed on a tangible, non-transitory medium, such as a computer readable medium, for example, diskette, CD ROM, DVD, ROM, RAM, flash memory or hard disk. It could also comprise a series of computer readable instructions transmittable to a computer system, via a modem or other interface device, over either a tangible medium, including but not limited to optical or analogue communications lines, or intangibly using wireless techniques, including but not limited to microwave, infrared or other transmission techniques. The series of computer readable instructions embodies all or part of the functionality previously described herein.

Those skilled in the art will appreciate that such computer readable instructions can be written in a number of programming languages for use with many computer architectures or operating systems. Further, such instructions may be stored using any memory technology, present or future, including but not limited to, semiconductor, magnetic or optical, or transmitted using any communications technology, present or future, including but not limited to optical, infrared or microwave. It is contemplated that such a computer program product may be distributed as a removable medium with accompanying printed or electronic documentation, for example, shrink wrapped software, pre-loaded with a computer system, for example, on a system ROM or fixed disk, or distributed from a server or electronic bulletin board over a network, for example, the Internet or World Wide Web.

Thus, viewed from a third aspect, the present invention provides a computer program product or a tangible computer-readable medium storing a computer program product, the computer program product comprising computer readable instructions that, when executed by a computer, will cause the computer to perform a method as set out above.

Viewed from a fourth aspect, the present invention provides a computer comprising: a memory, and a processor, the memory storing computer readable instructions that, when executed by the processor will perform a method as set out above.

Embodiments of the present invention will now be described in greater detail, by way of example only, and with reference to the accompanying figures, in which:

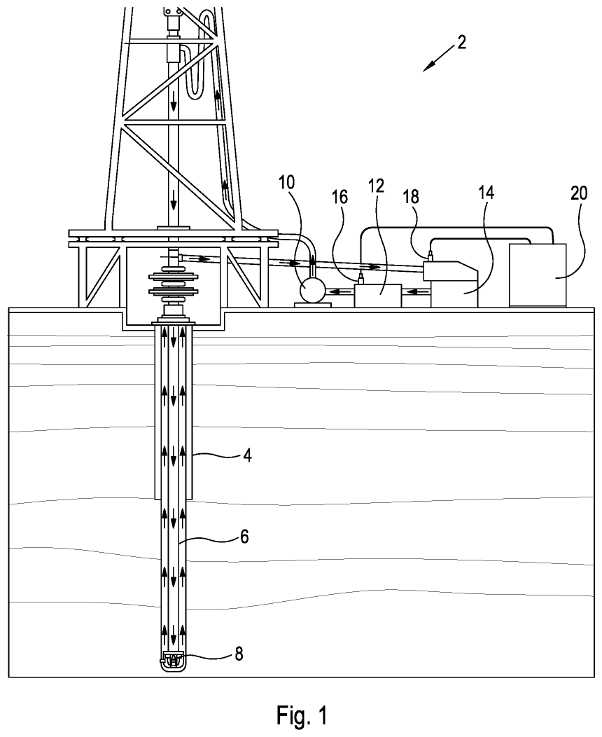

FIG. 1 shows a well drilling apparatus;

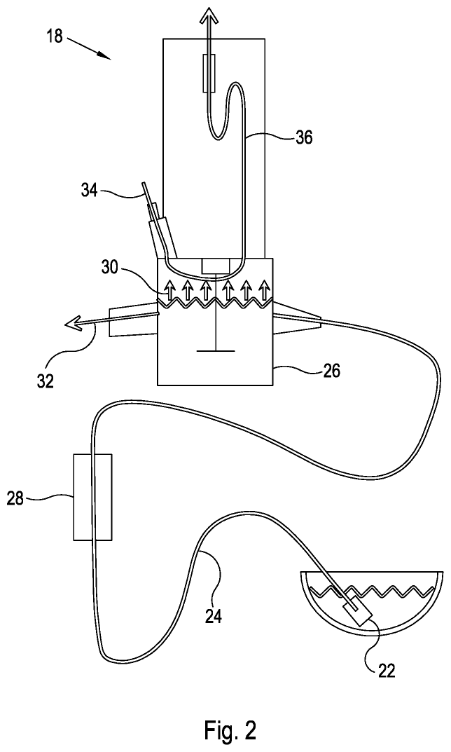

FIG. 2 shows a mud degasser for use with the well drilling apparatus;

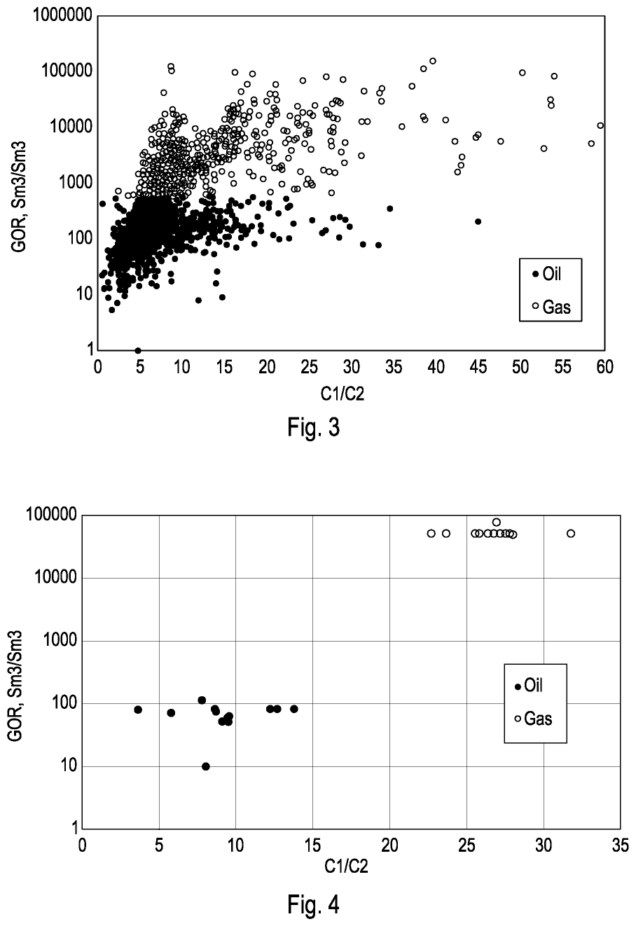

FIG. 3 shows a plot of C₁/C₂ ratio against gas-oil ratio for gas and oil reservoirs from a plurality of different fields;

FIG. 4 shows a plot of C₁/C₂ ratio against gas-oil ratio for gas and oil reservoirs from a single field;

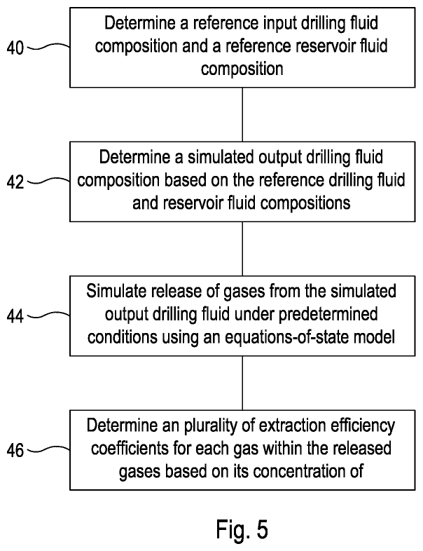

FIG. 5 shows a flow chat for a method of determining an extraction efficiency coefficient;

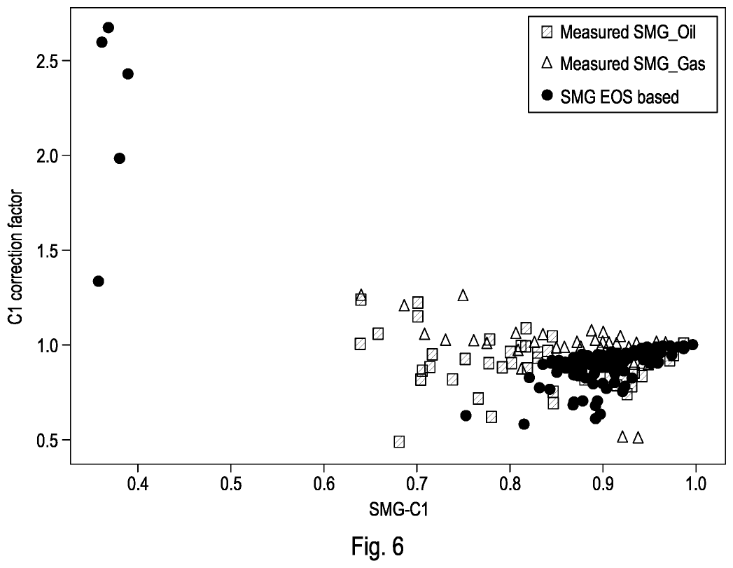

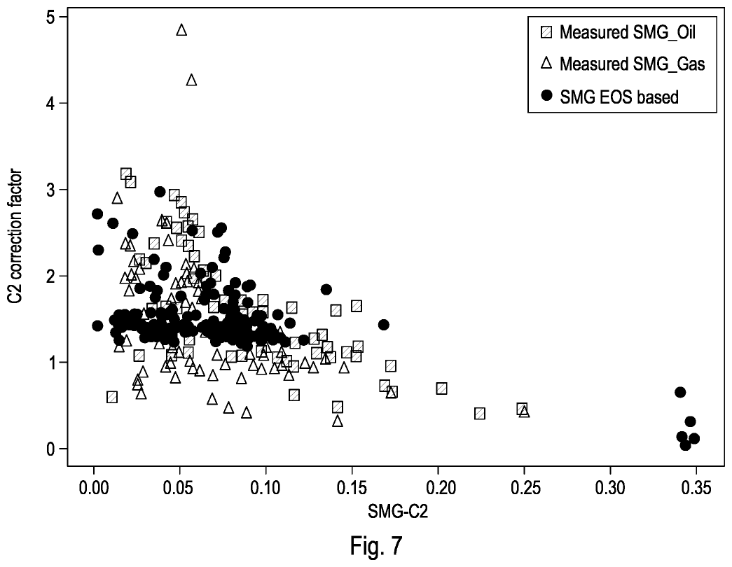

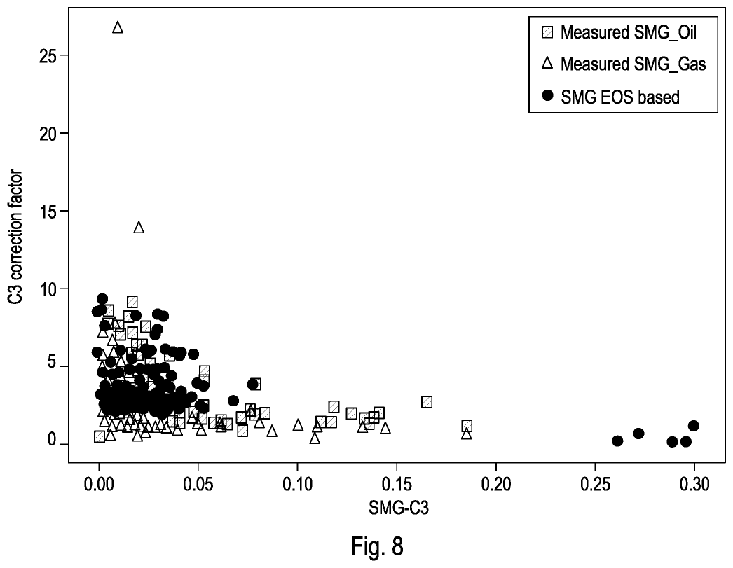

FIGS. 6 to 8 shows a comparison between empirically derived extraction efficiency coefficients, and extraction efficiency coefficients derived by the method shown in FIG. 5;

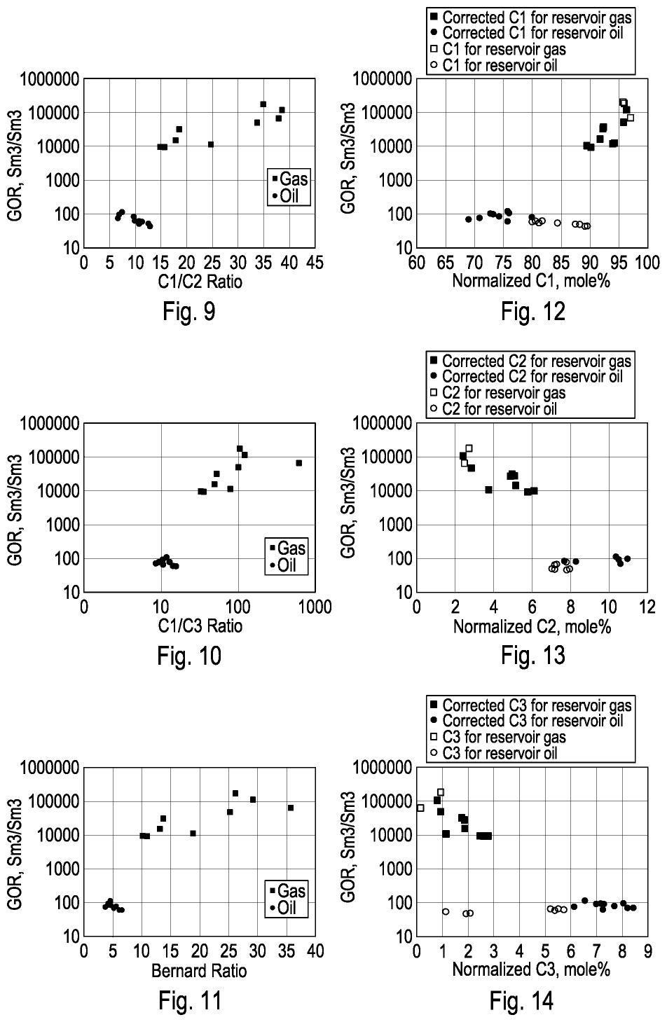

FIGS. 9 to 11 show plots of three different geochemical parameters against gas-oil ratio for gas and oil reservoir fluid samples;

FIGS. 12 to 14 show plots of C₁, C₂ and C₃ hydrocarbon concentrations, respectively, against gas-oil ratio for gas and oil reservoir fluid samples before and after correction; and

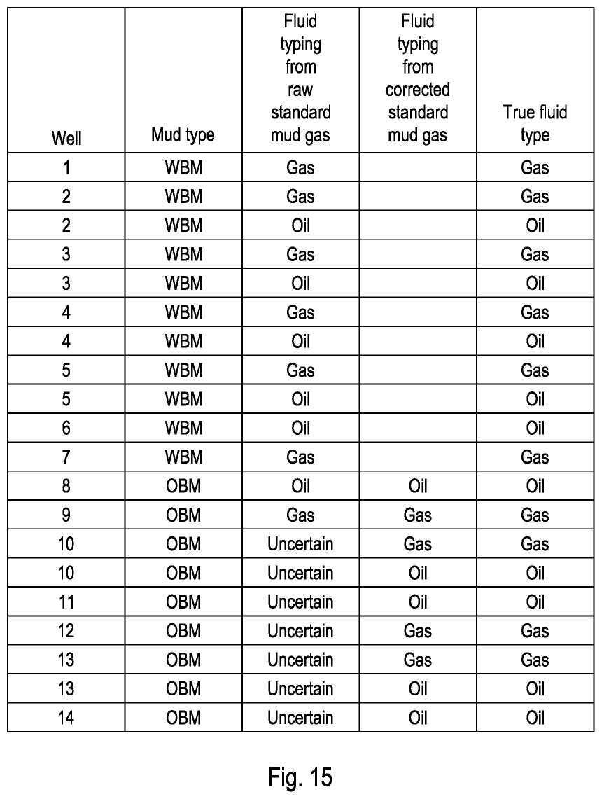

FIG. 15 shows a table illustrating the predicted and measured fluid types of a plurality of wells.

FIG. 1 shows a well drilling apparatus 2 for drilling a well bore 4. The illustrated apparatus 2 is for onshore well drilling. However, the techniques discussed herein are applicable also to offshore well drilling.

The apparatus 2 comprises a drill pipe 6 connected to a drill bit 8. The drill pipe 6 transmits torque and drilling fluid to the drill bit 8. Drilling fluid is supplied by a pump 10 from a mud pit 12 to the drill pipe 6. The pump 10 is usually a positive displacement pump to facilitate supply of high volumes of drilling fluid at high pressures. The mud pit 12 usually comprises a series of large, steel tanks that hold the drilling fluid for injection into the well bore 4.

Drilling fluids are broadly categorised into water-based drilling fluid, non-aqueous drilling fluid, also referred to as oil-based drilling fluid, and gaseous drilling fluid. Liquid drilling fluid, i.e. water-based drilling fluid and non-aqueous drilling fluid, are commonly referred to as “drilling mud”.

The drilling fluid serves to cool and lubricate the drill bit 8, and to carry cuttings from the drill bit 8 out of the well bore 4. After passing through or around the drill bit 8, the drilling fluid passes back up the well bore 4, outside of the drill pipe 6. The drilling fluid can also provide hydrostatic pressure to prevent formation fluids from entering into the well bore 4, as well as carrying out drill cuttings and suspending the drill cuttings while drilling is paused and when the drilling assembly is brought in and out of the well bore 4.

At the surface, the drilling fluid from the well bore 4 is passed through a solids control apparatus 14 for removal of solid material from the drilling fluid. The solids control apparatus 14 may comprise any one or more of a shale shaker, a desander, a desalinator and a desilter. After passing through the solids control apparatus 14, the drilling mud is returned to the mud pit 12 for reuse.

A mud-gas analysis unit 20 is provided for collection of mud-gas data. The mud-gas analysis unit 20 is connected to at least an output mud degasser 18 for the collection of mud gas from the drilling fluid after passing through the well bore 4. In some cases, e.g. when collecting “advanced mud-gas data”, the mud-gas analysis unit 20 may additionally be connected to an input mud degasser 16 for the collection of mud gas from the drilling fluid before passing through the well bore 4.

In the illustrated embodiment, the input mud degasser 16 samples drilling fluid from the mud pit 12, and the output mud degasser 18 samples drilling fluid from the solids control apparatus 14. However, it will be appreciated that the mud degassers 16, 18 may be positioned at other locations upstream and downstream of the well bore 4, respectively.

FIG. 2 shows, schematically, further details of the output mud degasser 18. The configuration of the input mud degasser 16, if used, is substantially the same.

The mud degasser 18 comprises a sampling probe 22 disposed so as to collect a sample 24 of the drilling mud from a source of drilling fluid. The drilling fluid sample 24 may be taken from a continuously flowing sample, e.g. such as a portion of a flow of drilling fluid within a flow line that is diverted through the mud degasser 18, or may be pumped from a static source of drilling fluid, such as the mud pit 12.

The drilling fluid sample 24 is supplied to a gas-separation chamber 26 where at least a portion of the gas carried by the drilling fluid is released. The sample of drilling fluid 24 may optionally be heated by a heater 28 upstream of the gas-separation chamber 26. Heating the drilling fluid sample 24 helps to release the gas from the drilling mud sample 24, particularly heavier hydrocarbon gases. Typically, the drilling fluid sample 24 is heated to a temperature of above 50° C., and sometimes to a temperature of around 80° C. to 90° C.

The released gas 30 is directed from the separation chamber 26 to the gas analysis unit 20, while the degassed mud 32 is returned to the flow line or to another location for re-use.

A carrier gas stream 34, commonly comprising air, may be supplied to the separation chamber 26 and mixed with the released gas 30 to form a gas mixture 36 that is supplied to the mud-gas analysis unit 20. The carrier gas stream 34 provides a continuous flow of carrier gas in order to provide a substantially continuous flow rate of the gas mixture 36 from separation chamber 26 to the mud-gas analysis unit 20. Additionally, in the case of a mud-gas analysis unit 20 comprising a combustor, the use of air as the carrier gas may provide the necessary oxygen for combustion.

The mud-gas analysis unit 20 comprises a device suitable for detailed analysis of the composition of the hydrocarbon gas mixture. This analysis is usually performed by a gas chromatograph. However, other detecting devices may also be utilised including a mass spectrometer, an infrared analyser or a thermal conductivity analyser.

A gas chromatograph is a rapid sampling, batch processing instrument that provides a proportional analysis of a series of hydrocarbons. Gas chromatographs can be configured to separate almost any suite of gases, but typically oilfield chromatographs are designed to separate the paraffin series of hydrocarbons from methane (C₁) through pentane (C₅) at room temperature, using air as a carrier. The chromatograph will report (in units or in mole percent) the quantity of each component of the gas detected.

In some arrangements, the mud-gas analysis unit 20 may be configured to detect and/or remove H₂S from the gas to prevent adverse effects that could influence hydrocarbon detection.

In some embodiments, non-combustibles gases, such as helium, carbon dioxide and nitrogen, can be detected by the mud-gas analysis unit 20 in conjunction with the logging of hydrocarbons.

The most common gas component present in mud gas is usually methane (C₁). The presence of heavier hydrocarbons, such as C₂ (ethane), C₃ (propane), C₄ (butane) and C₅ (pentane) may indicate an oil or a “wet” gas zone. Even heavier molecules, up to about C₇ (heptane) or C₈ (octane), may also be detectable, but are typically present only in very low concentrations. Consequently, the concentrations of these hydrocarbons are often not recorded.

The measured composition of the mud gas is usually referred to as “raw” mud-gas data and is not comparable to the actual composition of the reservoir fluid, since the mud gas contains gases that do not originate from the reservoir (e.g. gases present in the drilling mud or remaining from previous injection when recycling the drilling mud) and also because lighter hydrocarbon (e.g. C₁) are carried/released more easily by the drilling mud than heavier hydrocarbons (e.g. C₂ to C₅).

Depending on how the mud-gas data is collected and processed, it is normally described as being one of two types of mud-gas data.

“Standard mud-gas data” is raw mud-gas data, which is usually collected without the use of a heater 28. Standard mud-gas data usually includes measurements of the C₁ to C₅ composition. However, the lack of heating means that standard mud-gas data has limited gas components that can be detected confidently (usually from C₁ to C₃). Standard mud-gas data is routinely collected when drilling most well bores 4, and most well bore drilling apparatuses 2 include a standard mud-gas analysis unit 20.

“Advanced mud-gas data” also usually includes measurements of the C₁ to C₅ composition, but has a composition that more closely corresponds to that of the reservoir fluid. Advanced mud-gas data is less commonly collected when drilling well bores 4, and is usually collected by an external contractor who will temporarily install an advanced mud-gas analysis unit 20 whilst drilling the well bore 4, which is then removed afterwards.

When measuring advanced mud-gas data, the mud gas sample is usually collected with the use of a heater 28. Additionally, one or more corrections are made to the raw mud-gas data to more accurately predict the actual composition of the reservoir fluid. Two common corrections applied when measuring advanced mud-gas data are hydrocarbon recycling correction and extraction efficiency correction (EEC).

A recycling correction is made to eliminate contamination of the gas by gases originating from previous injections of the drilling mud. This correction is applied based a separate mud-gas measurement that was taken before the drilling mud was injected into the well bore 4, i.e. collected using an input mud degasser 16.

An extraction efficiency correction is made to modify the fractional concentration of the hydrocarbon components, such that the mud-gas data after this step closely resembles a corresponding reservoir fluid composition. The extraction efficiency correction comprises applying respective extraction efficiency coefficients to the fractional concentration of each of the components of the raw mud-gas data.

Reservoir fluid identification has traditionally been performed based on examination of various standard gas component ratios, known as geochemical parameters.

When using standard mud-gas data, these typically include C₁/C₂, C₁/C₃, and the Bernard parameter (C₁/C₂+C₃). Where advanced mud-gas data is available, this may further include the balance ratio (C₁+C₂/C₃+C₄+C₅), the wetness ratio (C₂+C₃+C₄+C₅/C₁+C₂+C₃+C₄+C₅), the dryness ratio (C₁/C₁+C₂+C₃+C₄+C₅), and the hydrocarbon character (C₄+C₅/C₃). Other geochemical parameters may additionally or alternatively be used.

For each component ratio, a universal threshold (e.g., 15 in the case of the C₁/C₂) has been used as a rule-of-thumb to divide reservoir oil and reservoir gas. That is to say, when C₁/C₂ ratio is higher than 15, the reservoir fluid is typically gas prone, and when the C₁/C₂ ratio is lower than 15, the reservoir fluid is typically oil-prone. FIG. 3 shows a plot containing data from approximately 4000 reservoir fluid samples taken across a large range of oil fields around the world. The plot correlates a gas-oil ratio of the fluid sample (y-axis) against a C₁/C₂ ratio of the fluid sample. The term gas-oil ratio refers to the ratio of the volume of gas that comes out of solution at surface conditions to the volume of oil under the same conditions. Green dots are used to indicate oil-phase fluid samples, and red dots are used to indicate gas-phase fluid samples. At reservoir conditions, the phase boundary between gas and oil occurs at a gas-oil ratio of approximately 600 Sm³/Sm³.

As will be apparent from FIG. 3, there is a significant overlap between gas and oil fluid samples in the C₁/C₂ ratio range of 5 to 20. Within this range, it is not possible to confidently distinguish between oil and gas fluid samples based only on the C₁/C₂ ratio. However, a large number of reservoirs have fluid compositions falling within this range, which is the reason why the previous empirical correlations cannot be relied upon.

Whilst only the C₁/C₂ ratio is shown here, other geochemical parameters can also be used to distinguish between gas phase and oil phase fluids, and similar empirical correlations exist for many of these parameters. The empirical correlations for these other parameters also suffer from the same issue of low accuracy.

The present inventors have examined the reservoir composition data and identified that, although there is a great degree of overlap between geochemical parameters when considered globally, there is often a much clearer separation between the geochemical parameter values for gas and oil fluid samples within individual reservoirs or oil fields.

Specifically, within a single hydrocarbon basin, the reservoir oils and gases are often from the same origin. Consequently, by considering only the specific oils and gases from a single basin, it is often possible to easily distinguish these specific fluids based on their geochemical parameters.

FIG. 4 illustrates 25 fluid samples taken from a single field. The oil samples are shown in green, and the gas samples are shown in red. As can be seen, in this particular field, all of the oil samples have a C₁/C₂ ratio of below 14, whilst all of the gas samples have a C₁/C₂ ratio of greater than 22.

Based on this data, a region-specific C₁/C₂ ratio threshold can be determined for distinguishing between oil and gas within this reservoir, for example of about 18. This region-specific threshold can then be used when drilling new wells within the same field, similar to how the global thresholds were used in the past. However, significantly, a much greater confidence can be associated with that threshold, thereby allowing decisions to be made based purely on this geochemical analysis.

This is advantageous because many geochemical parameters can be determined based on mud-gas data collected whilst drilling the reservoir. This data is comparatively cheap to collect, is also available at a very early stage of well drilling, and can be collected as a continuous log along the length of the well. Thus, it can inform decisions in real-time regarding how to place and complete the well, such as where to perforate the well casing.

Whilst FIG. 4 shows only a plot for the C₁/C₂ ratio of this reservoir, clear delineation between oil and gas fluid samples can also be present for other geochemical parameters.

As discussed above, whilst advanced mud-gas analysis is comparatively cheap compared to collecting a large number of downhole fluid samples, it nevertheless incurs a significant additional cost compared to collecting only standard mud-gas data. Therefore, if analysis can be performed using only standard mud-gas data, this would be highly advantageous.

Standard mud-gas data typically provides a reasonable approximation of the C₁, C₂ and C₃ composition of the reservoir fluid, particularly when using water-based drilling fluid. However, standard mud-gas data typically gives poor approximations for the C₄ and C₅ compositions of the reservoir fluid. This is because, without heating of the drilling mud, only very low quantities of these components are released.

This means that, if the region-specific thresholds associated with the C₁/C₂ ratio, the C₁/C₃ ratio or the Bernard parameter (C₁/C₂+C₃) exhibit sufficient confidence that they can be used to confidently differentiate between oil and gas reservoir fluid for a particular field, then standard mud-gas data can be used for this analysis without the need to collect costlier advanced mud-gas data. Specifically, when considering the degree of confidence required, a greater degree of confidence is required when using standard mud-gas data than when using advanced mud-gas data due to the lower accuracy of the data itself.

Fields where the C₁-C₃ composition is sufficient for fluid typing are described herein as Type I fields.

Should the analysis indicate that none of the C₁/C₂ ratio, the C₁/C₃ ratio or the Bernard parameter (C₁/C₂+C₃) exhibits sufficient confidence, then it will be necessary to collect more accurate information concerning the C₄ and C₅ composition in order to accurately distinguish between oil and gas within the reservoir.

Fields where the C₁-C₅ composition is required for fluid typing are described herein as Type II fields.

This C₄ and C₅ data can be obtained by collecting advanced mud-gas data. Alternatively, a more cost-effective option than collecting advanced mud-gas data may be to instead employ a heated standard mud-gas analysis tool (or to employ a single, advanced mud-gas analysis tool on only the drilling mud outlet to collect standard mud-gas data).

Normally, standard mud-gas analysis tools do not employ heating, but some more recent tools do employ heating, such as up to about 50° C. This is lower than is normally used when collecting advanced mud-gas data, where the analysis tool typically heads the drilling mud to temperatures of up to about 90° C. However, by employing these higher temperatures (either up to about 50° C., or about 90° C.) in the collection of standard mud-gas data, it is possible to increase the quantities of C₄ and C₅ hydrocarbons released from the drilling mud.

If the confidence associated with the relevant geochemical properties is sufficiently high, i.e. those requiring C₄ and C₅ measurements, it may be possible to use this data for the purposed of fluid typing, even though the data is not as accurate as advanced mud-gas data.

Whilst the analysis regarding whether or not C₄ and C₅ data is required may be determined based on the geochemical parameter threshold confidences determined for the particular region, the inventors have identified that low confidence associated with the C₁ to C₃ geochemical parameter thresholds commonly occur in situations where the reservoir gas is of high liquid yield (e.g. having a gas-oil ratio between 600 and 1200 Sm³/Sm³) and the reservoir oil is highly volatile (e.g. having a gas-oil ratio between 300 and 600 Sm³/Sm³). Consequently, a decision regarding whether or not C₄ and Cs data is required (i.e. whether the field is a type I field or a type II field) may be taken based on the compositions of the reservoir oil and the reservoir gas within the region of interest.

As discussed above, standard mud-gas data typically provides a good approximation of the C₁, C₂ and C₃ composition of the reservoir fluid. However, one limitation of standard mud-gas data is no extraction efficiency correction has been developed, such as is applied for advanced mud-gas data. The C₁-C₃ gas components have high fugacity, and the inventors have identified that the C₁-C₃ compositions in standard mud-gas is similar to those of reservoir fluid samples when water-based mud is used. However, when oil-based mud is used, the C₁-C₃ composition in standard mud-gas data differs from those of the reservoir fluid samples.

The following technique, illustrated in FIG. 5, provides an extraction efficiency correction method based on an Equation of State (EOS) simulation, which can be applied to standard mud-gas data.

This method is particularly beneficial when examining the composition of Type I fields, where only standard mud-gas data has been collected and where the well bores were drilled using oil-based drilling fluid. However, it may be applied more generally to other types of fields.

In step 40, a reference input drilling fluid composition and a reference reservoir fluid composition are determined.

The reference drilling fluid composition may be determined by taking measurements, for example of actual drilling fluid. Alternatively, a composition of the drilling fluid to be used may be supplied by a drilling fluid suppler. As mentioned above, this technique is particularly applicable where the drilling fluid is an oil-based drilling fluid.

In the case of determining well-specific extraction efficiency coefficients, the reference reservoir fluid sample may be taken from the specific well that the coefficients are targeting. The reservoir fluid samples may be collected using downhole fluid sampling techniques, such as wireline sampling. The fluid composition may be determined using in-situ testing, i.e. using downhole fluid analysis tools, or the reservoir fluid sample may be analyses in a testing laboratory.

Alternatively, in some cases, the composition of reservoir fluid is can be similar across an entire field. Based on exploration wells, appraisal wells, and production history, it may be possible to estimate fluid composition around a drilling target. Thus, in these cases, the reference reservoir fluid sample may be taken from nearby wells, for example an analog well within the same oil field. In this case, the reference reservoir fluid composition may be retrieved from a database of historical reservoir fluid samples collected from the oil field.

In step 42, a simulated output drilling fluid composition is determined.

The simulated output drilling fluid composition represents a predicted composition of a drilling fluid after having been circulated through the well bore 4 whilst the drill bit 8 is in operation.

Typically, during circulation through the well bore, the drilling fluid may absorb up to about 1 wt. % of the reservoir fluid. A typical gas response might be in the range of 50-1000 ppm. Therefore, in one embodiment, the simulated output drilling fluid composition may comprise a mixture of 99 wt. % of the reference drilling fluid composition and 1 wt. % of the reference reservoir fluid composition. However, other mixture ratios may also be used.

In step 44, the release of gases from the simulated output drilling fluid under predetermined operating condition is simulated.

The simulation is performed using an equations-of-state (EOS) model. An EOS is a fluid model that takes a molar composition of a fluid and predicts the phase and volumetric behaviour of the fluid (e.g., densities, viscosities and formation volume factors) over a range of pressures and temperature.

Using the EOS model, it is possible to determine a concentration of each hydrocarbon that is in a gaseous state under the predetermined conditions. The hydrocarbons in gaseous state represent a simulated (output) mud gas.

The predetermined conditions correspond to the expected conditions within the mud degasser 16. For example, the pressure may be approximately atmospheric pressure (e.g. 0.9 bar to 1.1 bar) and the temperature may be the heated temperature of the mud degasser 16 (e.g. 80° C. to 90° C.). However, other temperatures and/or pressures may be used. For example, in the case of a mud degasser 16 that does not use heating, the temperate may be anywhere between about 10° C. to about 50° C., depending on the specific drilling and reservoir conditions.

In step 46, extraction efficiency coefficients are determined.

Each extraction efficiency coefficient represents a multiplier to be applied to a respective fractional composition of measured mud gas. Each multiplier modifies a fractional composition such that its corrected value corresponds to the respective fractional composition of the reservoir fluid.

An extraction efficiency coefficient is normally determined for each fractional hydrocarbon component of the released gas. Typically, an extraction efficiency coefficient would be determined for at least the C₁ to C₃ fractions, and optionally also for the C₄ and C₅ fractions, or for any other fraction of the mud-gas data.

In order to determine the extraction efficiency coefficient for a selected hydrocarbon, Cₓ, the fractional composition of Cₓ is determined in the simulated mud-gas and in the reference reservoir fluid. Then, the reference reservoir fluid value is divided by the simulated mud-gas value to determine the extraction efficiency coefficient.

Thus, when the composition of real mud gas is measured, the Cₓ fractional composition of the measured mud-gas can be multiplied by the extraction efficiency coefficient to correct for extraction efficiency.

As will be appreciated, the techniques described above produce extraction efficiency coefficients. However, they do not require the use of measured mud-gas data, and instead require only knowledge of the drilling fluid composition and a reference reservoir fluid composition. These techniques therefore avoid problems associated with using measured mud-gas, as it can be affected by external factors, i.e. factors other than the reservoir fluid composition.

Furthermore, the extraction efficiency coefficients can be easily recalculated if it is desired to change the operating conditions of the mud degassers 16, 18. Specifically, recalculating the extraction efficiency coefficients in this case does not require taking new downhole reservoir fluid samples, which is a relatively expensive process.

The techniques described above may be implemented at least partially using software, e.g. computer programs.

FIGS. 6 to 8 show for each of C₁, C₂ and C₃, respectively, predicted and measured extraction efficiency coefficients for a plurality of reservoir fluid samples.

The measured extraction efficiency coefficient corresponds to a measured, Cₓ composition of the standard mud-gad data divided by a measured Cₓ composition of the reservoir fluid.

The predicted extraction efficiency coefficients are predicted by the method described above.

In each graph, the Cₓ concentration in the reservoir fluid is shown on the x-axis, and the measured or predicted Cₓ extraction efficiency coefficient is shown on the y axis.

In each graph, the measured extraction efficiency coefficients for reservoir oils are shown in green, the measured extraction efficiency coefficients for reservoir gases are shown in red, and the predicted extraction efficiency coefficients are shown in blue.

As can be seen, the predicted extraction efficiency coefficients for each of the C₁ to C₃ fractions show a good correlation with the measured extraction efficiency coefficients of both reservoir oils and reservoir gases. This indicates that this technique provides usable extraction efficiency coefficients.

A workflow to predict fluid typing along a well bore within a particular oil field using standard mud-gas data is therefore as follows:
1) Investigate a reservoir fluid database to define if the oil field belongs to Type I or Il.
2) If the field belongs to Type I, define the C₁/C₂, C₁/C₃, and Bernard parameter gas-oil thresholds for the oil field based on the reservoir fluid database.
3) Investigate if water-based mud or oil-based mud is used for drilling.
4) In the case of water-based mud, the C₁/C₂, C₁/C₃, and Bernard parameter gas-oil thresholds can be used to define fluid typing based on the standard mud-gas data without correction.
5) In the case of oil-based mud, determine and apply extraction efficiency coefficients to correct the raw standard mud-gas data to obtain the corrected standard mud-gas data. Then use the C₁/C₂, C₁/C₃, and Bernard parameter gas-oil thresholds to define fluid typing based on the corrected standard mud-gas data.

An example of the application of this workflow will now be described with reference to FIGS. 9 to 14.

Johan Castberg is an oil field located in the Barent Sea consists of the three discoveries Skrugard, Havis, and Drivis. The reservoir fluids have gas caps with lean gas and oil legs with black oil.

FIGS. 9 to 11 show, respectively, plots of the C₁/C₂ ratio, C₁/C₃ ratio, and Bernard parameter for reservoir fluid samples taken from different wells within the Johan Castberg field.

For each plot, the geochemical parameter is shown on the x-axis, and the gas-oil ratio is shown in the y-axis. Fluid samples from gas reservoirs are shown in red, and fluid samples from oil reservoirs are shown in green.

These plots illustrate a clear divide between oil and gas samples. The C₁/C₂ ratio threshold is about 13, the C₁/C₃ ratio threshold is about 25, and the Bernard parameter threshold is about 8.

Therefore, the Johan Castberg field is a Type I field.

Next, extraction efficiency coefficients were derived for the C₁-C₃ components in the standard mud gas. An EOS-based flash simulation was performed with specific data from the Johan Castberg field, including the drilling fluid composition, the reservoir oil and gas sample compositions, and the mud degasser temperature and pressure ranges.

FIGS. 12 to 14 show, respectively, the C₁, C₂ and C₃ compositions from standard mud-gas data samples before and after correction.

For each plot, the normalized Cₓ mud-gas composition is shown in the x-axis and the gas-oil ratio is shown in the y-axis. The raw mud-gas data values are shown as hollow circles, and the corrected mud-gas data values are shown as solid circles. Again, fluid samples from gas reservoirs are shown in red, and fluid samples from oil reservoirs are shown in green.

FIG. 12 shows that normalized C₁ composition was reduced both for oil and gas samples. On the contrary, the normalized C₂ and C₃ compositions were increased, as shown in FIGS. 13 and 14.

It is noted particularly that the correction C₃ composition demonstrates a much clearer separation for oil and oil samples, which helps significantly for accurate fluid typing.

FIG. 15 shows the output of the reservoir fluid typing analysis performed on 14 wells using mud-gas analysis, as compared against the true reservoir fluid type determined using downhole fluid analysis.

During exploration and appraisal phases, seven wells (wells 1-7) were drilled using water-based mud (WBM). Therefore, the C₁-C₃ component ratio thresholds, derived from the reservoir fluid database, could be applied directly to this mud-gas data, i.e. without correction. Satisfactory fluid typing results were achieved for all 7 exploration and appraisal wells from the standard mud-gas data when water-based mud was used (as shown in FIG. 15).

The standard mud gas data for wells 1-7 was provided from two different vendors. This fact adds even more confidence for implementing the field-specific gas component ratio thresholds for fluid typing.

Additionally, the gas component ratios for C₁/C₂, C₁/C₃, and Bernard parameter determined using mud-gas data were compared with corresponding rations determined using downhole fluid samples at four sampling depths. The data showed an excellent agreement in all three ratios between the raw standard mud-gas data and the reservoir fluid samples. This supports that the C₁-C₃ composition in standard mud-gas data can be used directly without correction when water-based mud is used.

When production drilling started, the drilling fluid was switched to oil-based mud (OBM). A further seven production wells (wells 8-14) were drilled using the oil-based drilling fluid.

Fluid typing was attempted using both the raw standard mud-gas data, as well as corrected standard mud-gas data, where the calculated extraction efficiency corrections were applied to correct the raw standard mud-gas data.

As can be seen from FIG. 15, the raw standard mud-gas data was insufficient to correctly identify the reservoir fluid type alone. However, excellent results were achieved for all wells when the extraction efficiency corrections were applied.

1. A method of calculating an extraction efficiency coefficient for mud-gas analysis, the method comprising:
providing a composition of an input drilling fluid;
providing a composition of a reservoir fluid;
generating a composition of a simulated output drilling fluid based on the compositions of the input drilling fluid and the reservoir fluid;
simulating release of a selected gas component from the simulated output drilling fluid under predetermined conditions; and
determining the extraction efficiency coefficient for the selected gas component based on a ratio between a concentration of the selected gas component within the composition of the reservoir fluid and a simulated concentration of the selected gas component released from the simulated output drilling fluid.

2. The method according to claim 1, wherein simulating release of the selected gas component from the simulated output drilling fluid under predetermined conditions is performed using an equations-of-state model corresponding to the composition of the simulated output drilling fluid.

3. The method according to claim 1, wherein the extraction efficiency coefficient corresponds to a specific hydrocarbon field, and wherein the composition of the reservoir fluid is a composition of a reservoir fluid sample from a hydrocarbon well in the specific hydrocarbon field.

4. The method according to claim 1, wherein the extraction efficiency coefficient corresponds to a specific hydrocarbon well, and wherein the composition of the reservoir fluid is a composition of a reservoir fluid sample from the specific hydrocarbon well.

5. The method according to claim 1, wherein the predetermined conditions correspond to operational conditions of a mud-gas analysis unit.

6. The method according to claim 1, wherein the simulated output drilling fluid comprises between 0.01 wt. % and 5 wt. % of the reservoir fluid and a balance of the input drilling fluid.

7. A method comprising:
receiving mud-gas data; and
performing an extraction efficiency correction on the mud-gas data to produce corrected mud-gas data,
wherein the extraction efficiency correction comprises applying a plurality of extraction efficiency coefficients to the mud-gas data, each extraction efficiency coefficient having been determined by the method according to claim 1.

8. The method according to claim 7, further comprising:
identifying a geochemical parameter based on the corrected mud-gas data, the geochemical parameter being derivable from C₁ to C₃ fluid composition data; and
identifying a fluid type of a target reservoir fluid based on a threshold associated with the geochemical parameter.

9. The method according to claim 8, where the geochemical parameter comprises one of:
a C₁/C₂ ratio;
a C₁/C₃ ratio; and
a Bernard parameter, C₁/(C₂+C₃).

10. The method according to claim 9, wherein the threshold is a region-specific threshold.

11. The method according to claim 10, further comprising:
obtaining reservoir fluid properties data corresponding to a plurality of fluid samples;
identifying a fluid type and a geochemical parameter for each of the fluid samples that are within the region of interest, the geochemical parameter being derivable from C₁ to C₃ fluid composition data;
determining the region-specific threshold for the geochemical parameter based on the fluid type of the plurality of fluid samples within the region of interest, the region-specific threshold being for distinguishing between a first fluid type and a second fluid type within the region of interest.

12. The method according to claim 7, further comprising:
drilling a well bore using drilling fluid, the well bore passing through a reservoir containing the reservoir fluid;
collecting output mud gas from the drilling fluid after passing through the well bore; and
measuring a composition of the output mud gas as the mud-gas data.

13. A computer program or a tangible computer-readable medium storing a computer program, the computer program comprising computer readable instructions that, when executed by a computer, will cause the computer to perform the method according to claim 1.

14. A computer program or a tangible computer-readable medium storing a computer program, the computer program comprising computer readable instructions that, when executed by a computer, will cause the computer to perform a method according to claim 7.
